(19) United States
(12) Patent Application Publication
(10) Pub. No.: US 2024/0190013 A1
(43) Pub. Date: Jun. 13, 2024

(54) METHOD AND DEVICE FOR PROVIDING FOOD INTAKE SUPPORT SERVICE

(71) Applicant: HYODOL CO., LTD., Anyang-si Gyeonggi-do (KR)

(72) Inventors: Jihee KIM, Seongnam-si Gyeonggi-do (KR); Se Jin CHUNG, Guri-si Gyeonggi-do (KR); Kwang Yu KIM, Hwaseong-si Gyeonggi-do (KR); Tae Yang OH, Guri-si Gyeonggi-do (KR); Dong Myoung WOO, Guro-gu Seoul (KR)

(21) Appl. No.: 18/444,464

(22) Filed: Feb. 16, 2024

Related U.S. Application Data
(63) Continuation of application No. PCT/KR2023/002284, filed on Feb. 16, 2023.

(30) Foreign Application Priority Data
Mar. 2, 2022 (KR) 10-2022-0027044

(51) Int. Cl. B25J 11/00; A61B 5/00; A61B 5/16; G16H 20/60

(52) U.S. Cl. CPC B25J 11/0005; A61B 5/165; A61B 5/4803; B25J 11/0045; G16H 20/60

(57) ABSTRACT

A method of providing a food intake support service includes outputting, by a second device included in a robot device comprising the second device and a first device, an alarm when a preset food intake time arrives, receiving, by the second device, a plurality of sensing data related to food intake from the first device, analyzing, by the second device, the plurality of sensing data; outputting, by the second device, feedback based on the plurality of sensing data and performing, by the second device, a real-time interactive communication based on inputted voice data.

### CROSS REFERENCE TO RELATED APPLICATIONS

This application is a continuation of International Patent Application No. PCT/KR2023/002284, filed on Feb. 16, 2023, which claims the priority to and benefits of Korean Patent Application 0-2022-0027044, filed on Mar. 2, 2022. All of the aforementioned patent applications are hereby incorporated by reference in their entireties.

### TECHNICAL FIELD

The present disclosure relates to a method and a device for providing a food intake support service.

### BACKGROUND

Due to the rapid aging of the population in the Republic of Korea, the percentage of elderly people aged 65 and above was 20.9% as of 2019, and is projected to rise to 20.3% in 2025 and 46.5% in 2067. In particular, according to the 2017 Senior Citizen Survey conducted by the Ministry of Health and Welfare in the Republic of Korea, only 19.6% of elderly households living alone responded that they had no difficulties in their daily lives. It has been found that the major difficulties experienced by elderly people living alone are the lack of people to care for them when they are sick and the constant feeling of psychological anxiety.

The increase in the elderly population has led to a demand for elderly care services, yet the reality is that there is a shortage of funding and manpower to provide care services. Recently, technologies for home smart healthcare devices, companion robots, and the like that support smart healthcare and self-health management have been developed to address this issue.

### SUMMARY

Embodiments of the present disclosure designed to resolve the conventional problems and provide a method and a device for providing a food intake support service that can conduct a natural conversation with a user while providing feedback to the user by checking the food intake status of the user.

The technical objects in accordance with the technical idea of the technology disclosed herein are not limited to the objects described above, and other objects that have not been mentioned will be clearly understood by those having ordinary skill in the art from the description below.

In one aspect, a method of providing a food intake support service in accordance with an embodiment of the present disclosure comprises the steps of: outputting, by a second device out of a first device and the second device included in a robot device, an alarm when a preset food intake time arrives; receiving, by the second device, a plurality of sensing data related to food intake from the first device; analyzing, by the second device, the plurality of sensing data; outputting, by the second device, feedback based on the plurality of sensing data; and performing, by the second device, real-time interactive communication based on inputted voice data.

In some implementations, the method further comprises the steps of: after the step of outputting the alarm, obtaining, by the second device, sensing data on a user who uses the robot device; and checking, by the second device, a mood of the user based on the sensing data obtained by the second device.

In some implementations, the step of performing the real-time interactive communication is a step of performing the real-time interactive communication based on the mood of the user checked based on the sensing data obtained by the second device and the voice data.

In some implementations, the step of receiving the plurality of sensing data is a step of receiving the plurality of sensing data measured from a plurality of sensors including at least one of a motion sensor, a salinity sensor, a gas sensor, or a temperature sensor provided in the first device that is a cutlery including a spoon.

In some implementations, the step of outputting the feedback is a step of outputting the feedback based on at least one of a mastication speed of the user, a salinity level contained in a food, whether the food is spoiled, or a temperature of the food based on the plurality of sensing data.

In some implementations, the method further comprises the steps of: checking, by the second device, whether the food intake has ended from the first device; and transmitting, by the second device, an analysis result obtained by analyzing the plurality of sensing data to a monitoring device.

In some implementations, the method further comprises the step of: after the step of checking whether the food intake has ended, creating and outputting, by the second device, a meal plan for a fixed period of time by taking a health condition of the user into account.

In some implementations, the method further comprises the steps of: after the step of creating and outputting the meal plan, receiving, by the second device, a purchase request signal for at least one of ingredients required for the meal plan from the user; and ordering ingredients according to the purchase request signal to a preset mart server.

In another aspect, a device configured to provide a food intake support service in accordance with an embodiment of the present disclosure comprises a first device comprising a plurality of sensors and configured to obtain a plurality of sensing data related to food intake, and a second device configured to output an alarm when a preset food intake time arrives, analyze the plurality of sensing data received from the first device and output feedback, and perform real-time interactive communication based on voice data inputted from the user.

In some implementations, the second device checks a mood of the user based on sensing data on the user and performs the real-time interactive communication based on the mood of the user and the voice data.

In some implementations, the first device is a cutlery comprising a spoon and is provided with the plurality of sensors comprising at least one of a motion sensor, a salinity sensor, a gas sensor, or a temperature sensor.

In some implementations, the second device outputs the feedback based on at least one of a mastication speed of the user, a salinity level contained in a food, whether the food is spoiled, or a temperature of the food based on the plurality of sensing data.

In some implementations, the second device transmits an analysis result obtained by analyzing the plurality of sensing data to a monitoring device upon checking from the first device whether the food intake has ended.

In some implementations, the second device creates and outputs a meal plan for a fixed period of time by taking a health condition of the user into account.

In some implementations, the second device orders ingredients according to a purchase request signal to a preset mart server in accordance with the purchase request signal for at least one of ingredients required for the meal plan received from the user.

In some implementations, the method and device for providing a food intake support service in accordance with the present disclosure can provide the health care service of users more conveniently and accurately without requiring manpower for care services by allowing for a natural conversation with a user while providing feedback to the user by checking the food intake status of the user.

The foregoing effects are merely examples, and effects predicted or expected from the detailed configuration of the present disclosure from the perspective of those having ordinary skill in the art may also be added to the unique effects of the present disclosure.

### BRIEF DESCRIPTION OF THE DRAWINGS

FIG. 1 is a diagram showing a system that provides a food intake support service in accordance with an embodiment of the present disclosure;

FIG. 2 is a diagram showing a robot device that provides a food intake support service in accordance with an embodiment of the present disclosure;

FIG. 3 is a diagram showing the main configuration of a second device included in a robot device that provides a food intake support service in accordance with an embodiment of the present disclosure;

FIG. 4 is a flow diagram for describing a method of providing a food intake support service in accordance with an embodiment of the present disclosure;

FIG. 5 is a flowchart for describing a method of providing a food intake support service by a second device included in a robot device in accordance with an embodiment of the present disclosure;

FIG. 6 is a diagram showing a system that provides a food intake support service in accordance with another embodiment of the present disclosure; and

FIG. 7 is a flowchart for describing a method of providing a food intake support service by a second device included in a robot device in accordance with another embodiment of the present disclosure.

### DETAILED DESCRIPTION

Hereinafter, embodiments disclosed herein will be described in detail with reference to the accompanying drawings, and identical or similar components will be assigned the same reference numerals regardless of the drawing symbols, and repetitive descriptions thereof will be omitted. The words “module” and “unit” for components used in the following description are given or used interchangeably solely taking into account the case of drafting the specification, and do not have distinct meanings or roles on their own. Further, in describing the embodiments disclosed herein, if it is determined that detailed descriptions of related known technologies would obscure the subject matter of the embodiments disclosed herein, the detailed descriptions thereof will be omitted. In addition, it should be understood that the accompanying drawings are intended only for easy understanding of the embodiments disclosed herein, the technical idea disclosed herein is not limited by the accompanying drawings, and all modifications, equivalents, and alternatives that fall within the spirit and technical scope of the present disclosure are included.

Terms including ordinal numbers, such as first, second, and the like, may be used to describe various components, but the components are not limited by such terms. These terms are used only for the purpose of distinguishing one component from another.

When a component is said to be “coupled” or “connected” to another component, it should be understood that it may be directly coupled or connected to said another component, but there may also exist other components in between. On the other hand, when a component is said to be “directly coupled” or “directly connected” to another component, it is to be understood that there are no other components in between.

Singular expressions include plural expressions unless the context clearly dictates otherwise.

It should be understood that terms such as “comprise” or “have” in the present application are intended to designate the presence of features, numbers, steps, operations, components, parts, or combinations thereof described in the specification, and not to preclude the possibility of the presence or addition of one or more other features, numbers, steps, operations, components, parts, or combinations thereof.

It will be appreciated that although the present disclosure has been illustrated and described in detail in the drawings and the foregoing description, the disclosure is to be considered illustrative in nature and not restrictive, only certain embodiments have been shown and described, and all changes and modifications that fall within the spirit of the disclosure are desired to be protected.

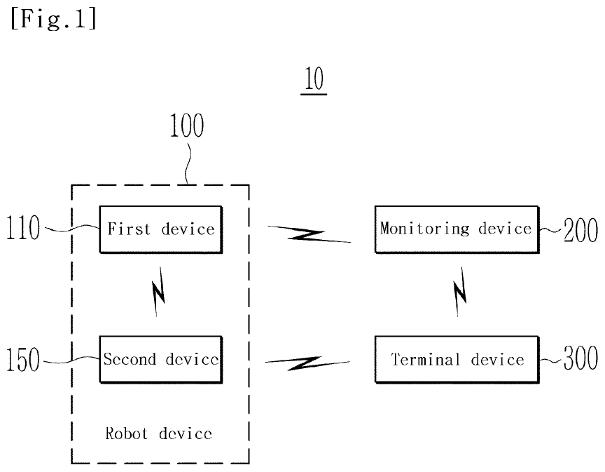

FIG. 1 is a diagram showing a system that provides a food intake support service in accordance with an embodiment of the present disclosure.

Referring to FIG. 1, a system 10 in accordance with the present disclosure may include a robot device 100, a monitoring device 200, and a terminal device 300.

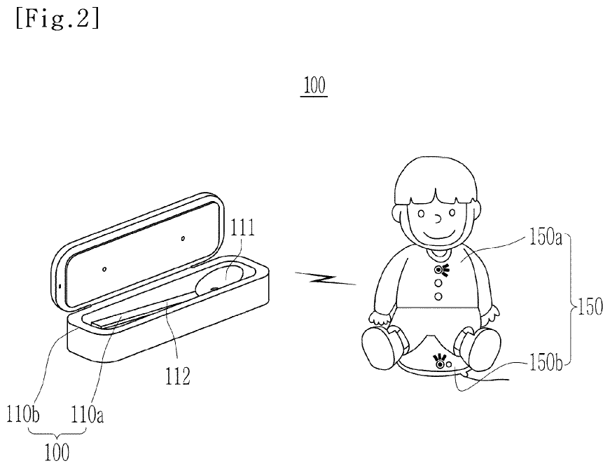

The robot device 100 may include a first device 110 and a second device 150, and the first device 110 and the second device 150 will be described in greater detail using FIG. 2 below. FIG. 2 is a diagram showing a robot device that provides a food intake support service in accordance with an embodiment of the present disclosure.

Referring to FIG. 2, a robot device 100 in accordance with the present disclosure may include a first device 110 and a second device 150, and the first device 110 may be a cutlery including a spoon that can move food to the mouth of a user. In addition, the second device 150 may be implemented in the form of a plush doll so as to provide a sense of closeness to the user.

The first device 110 may be implemented broadly as a spoon 110a and a spoon storage case 110b, and the spoon storage case 110b may include a charging unit to perform charging of the spoon.

The spoon 110a may include a plurality of sensors 111 and an LED 112, and may include a wireless charging unit (not shown) that can be placed in the storage case 110b and perform charging. In addition, the plurality of sensors 111 may include at least one of a salinity sensor, a temperature sensor, a gas sensor, or a motion sensor.

The salinity sensor may be or include a sensor that can sense the salinity of the food placed on the spoon 110a, the temperature sensor may be or include a sensor that can sense the temperature of the food placed on the spoon 110a, the gas sensor may be or include a sensor that can sense gases emitted from the food placed on the spoon 110a, and the motion sensor may be or include a sensor that can sense the movement of the spoon 110a. In addition, the spoon 110a may further include a sensor capable of sensing the acidity and oil content of the food.

The LED 112 may emit a green light if no abnormalities are detected in the food placed on the spoon 110a, and may emit a red light if any abnormality is detected in the food placed on the spoon 110a, for example, if the salinity is at or above a threshold, the temperature is at or above a threshold, or the concentration of gas discharged from the food is at or above a threshold. Thereby, the user can easily check the degree of abnormality of the food to be taken in via the LED 112 of the spoon 110a. The spoon 110a may store a threshold of salinity to be taken in per meal based on the health condition of the user, a threshold of a temperature suitable for intake by the user, and a threshold of gas concentration for checking the degree of spoilage of the food. In addition, the first device 110 mentioned below refers to the spoon 110a.

The second device 150 may include a robot 150a in the form of a plush doll and a cradle 150b on which the robot 150a can be placed and that can charge the robot 150a. The robot 150a of the second device 150 can provide a food intake support service by collecting a plurality of sensing data related to food intake measured from the first device 110 via communication with the first device 110 and providing feedback related to food intake to the user based on analysis results of the plurality of sensing data collected. To this end, the robot 150a may perform short-range wireless communication with the first device 110, such as Bluetooth, Bluetooth low energy (BLE), near field communication (NFC), or Zigbee.

Further, the robot 150a may analyze the mood of the user based on sensing data on the user. The robot 150a may perform real-time interactive communication with the user by outputting response data that takes into account the mood of the user analyzed based on the sensing data and voice data by the utterance of the user. In addition, the second device 150 mentioned below refers to the robot 150a.

The second device 150 performs communication with the monitoring device 200 and the terminal device 300. Thereby, the second device 150 may transmit the analysis results of the plurality of sensing data received from the first device 110 to at least one of the monitoring device 200 and the terminal device 300. To this end, the second device 150 may perform wireless communication with the monitoring device 200 and the terminal device 300, such as 5G (5th generation communication), LTE (long term evolution), LTE-A (long term evolution-advanced), or Wi-Fi (wireless fidelity).

The monitoring device 200 may be a device managed by a supplier that supplies the robot device 100, and may be a device such as a computer or server. The monitoring device 200 may store personal information including the health conditions of all users who purchased the robot device 100. The monitoring device 200 may create and provide a meal plan suitable for each of the users based on each personal information, and may set and provide intake salinity, intake temperature, and the like suitable for each of the users to the robot device 100.

The monitoring device 200 receives the analysis results of the plurality of sensing data obtained by the first device 110 via communication with the second device 150. The monitoring device 200 may visualize the average salinity value and average temperature value of the food taken in by the user, the eating time and the number of meals for the user, an AI statistical analysis report, and the like based on the received analysis results, and display them as visual data. The monitoring device 200 may transmit the visual data to the terminal device 300 of a caregiver or welfare worker of the user who uses the robot device 100.

In addition, the monitoring device 200 can diagnose depression and dementia in the user at an early stage based on the sensing data obtained by the second device 150 during interactive communication between the second device 150 and the user, and transmit it to the terminal device 300.

To this end, the monitoring device 200 may perform wireless communication with the second device 150 and the terminal device 300, such as 5G (5th generation communication), LTE (long term evolution), LTE-A (long term evolution-advanced), or Wi-Fi (wireless fidelity).

The terminal device 300 is a device used by the caregiver or welfare worker of the user who uses the robot device 100, and may be an electronic device, such as a smartphone, a tablet PC, or a computer. The terminal device 300 may download and install an application related to the food intake support service provided by the monitoring device 200.

The terminal device 300 may visualize the average salinity value and average temperature value of the food taken in by the user, the eating time and the number of meals for the user, an AI statistical analysis report, and the like based on the analysis results received from the second device 150 included in the robot device 100, and display them as visual data in the application. Further, the terminal device 300 may display, via the application, the visual data received from the monitoring device 200.

To this end, the terminal device 300 may perform wireless communication with the second device 150 and the monitoring device 200, such as 5G (5th generation communication), LTE (long term evolution), LTE-A (long term evolution-advanced), or Wi-Fi (wireless fidelity).

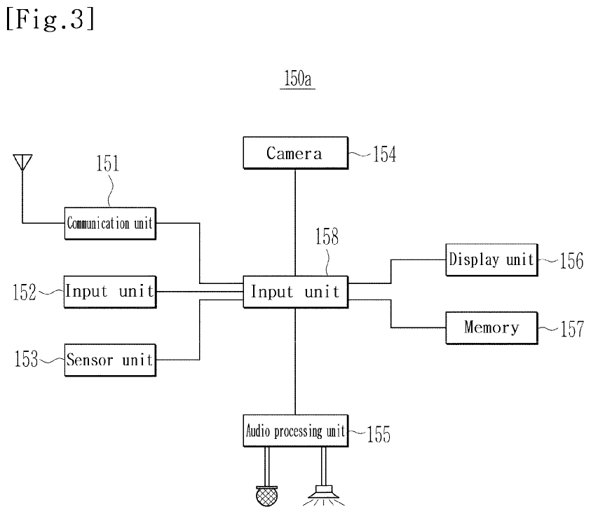

FIG. 3 is a diagram showing the main configuration of a second device included in a robot device that provides a food intake support service in accordance with an embodiment of the present disclosure.

Referring to FIG. 3, a robot 150a (hereinafter referred to as a second device 150) in accordance with the present disclosure may include a communication unit 151, an input unit 152, a sensor unit 153, a camera 154, an audio processing unit 155, a display unit 156, a memory 157, and a control unit 158.

The communication unit 151 performs communication with the first device 110. To this end, the communication unit 151 may perform short-range wireless communication, such as Bluetooth, Bluetooth low energy (BLE), near field communication (NFC), or Zigbee. In addition, the communication unit 151 performs communication with the monitoring device 200 and the terminal device 300. To this end, the communication unit 151 may perform wireless communication, such as 5G (5th generation communication), LTE (long term evolution), LTE-A (long term evolution-advanced), or Wi-Fi (wireless fidelity).

The input unit 152 generates input data in response to user input to the second device 150. To this end, the input unit 152 may include input devices such as a keyboard, a mouse, a keypad, a dome switch, a touch panel, touch keys, and buttons.

The sensor unit 153 may obtain sensing data on the movement of the user, the touch of the user, the body temperature of the user when the user touches the second device 150, and the like by including a radar sensor, a touch sensor, a temperature sensor, and the like. The sensor unit 153 may obtain sensing data including the heart rate of the user when the user holds the second device 150 for more than a certain period of time, such as embracing it in their arms or the like, by including a heart rate sensor.

The camera 154 may be an image sensor for obtaining image data on the user who uses the second device 150, and in particular, may obtain sensing data that is image data including the face of the user so that the facial expression of the user can be analyzed.

The audio processing unit 155 processes audio signals. In this case, the audio processing unit 155 includes a speaker and a microphone. The audio processing unit 155 plays audio signals outputted from the control unit 158 via the speaker, and the audio processing unit 155 transmits audio signals generated by the microphone to the control unit 158. In addition, the microphone may obtain the voice data of the user as sensing data.

The display unit 156 outputs output data according to the operation of the second device 150. To this end, the display unit 156 may include a display device, such as a flexible display, a liquid crystal display (LCD), a light emitting diode (LED) display, an organic light emitting diode (OLED) display, and the like. In addition, the display unit 156 may be combined with the input unit 152 and implemented in the form of a touchscreen.

Further, the display unit 156 may be disposed partially in the eye and lip regions of the facial portion of the second device 150, or may be disposed in the entire face region, and can display facial expressions and the like in a more three-dimensional way in the eye and lip regions so that the user can feel as if she or he were talking with a real person during interactive communication with the user. In particular, the display unit 156 can change and display facial expressions in the eye and lip regions according to the voice data outputted to the user based on the meaning contained in the voice data received from the user under the control of the control unit 158.

The memory 157 stores operation programs of the second device 150. The memory 157 may store time information for informing the user of a mealtime, a touch time for the second device 150, a time at which the second device 150 should sense the movement of the user, and the like.

The memory 157 may store an algorithm for analyzing a plurality of sensing data received from the first device 110, an algorithm capable of inferring the mood of the user by analyzing sensing data obtained by the sensor unit 153, the camera 154, and the microphone (hereinafter referred to as sensing data obtained by the second device 150), an AI chat algorithm that allows real-time interactive communication with the user, etc. In addition, the memory 157 may store connection addresses and the like of the monitoring device 200 and the terminal device 300 for transmitting the analysis results of the plurality of sensing data received from the first device 110 and the sensing data obtained by the second device 150.

When the arrival of a preset fixed time is confirmed, the control unit 158 outputs an alarm notifying this as voice data. In this case, the preset fixed time may include a mealtime, a touch time at which the robot device 100 should be touched, a time at which the robot device 100 should sense the movement of the user, and the like.

The control unit 158 may collect sensing data obtained from at least one of the first device 110 and the second device 150. At this time, if the mealtime has arrived, the control unit 158 may collect a plurality of sensing data obtained by the plurality of sensors 111 included in the first device 110, and may obtain sensing data from at least one of the sensor unit 153, the camera 154, and the microphone. Further, if the touch time or the time at which the movement should be sensed has arrived, the control unit 158 may obtain at least one of biosignal data including body temperature data of the user, movement data of the user, image data including the face of the user, and voice data of the user as sensing data by using at least one of the sensor unit 153, the camera 154, and the microphone.

The control unit 158 analyzes the obtained sensing data and performs the corresponding function. More specifically, the control unit 158 may check the mood of the user based on the sensing data obtained by the second device 150 when the mealtime has arrived. Further, the control unit 158 may check the salinity, temperature, whether the food is spoiled, etc., of the food to be taken in by the user based on the plurality of sensing data obtained by the first device 110, and may output feedback thereon as voice data.

In addition, the control unit 158 checks at least one of the body temperature, presence or absence of movement, and mood of the user based on the sensing data obtained by the second device 150 when the touch time or the time at which the movement should be sensed has arrived. Then, the control unit 158 may output feedback on the confirmed sensing data as voice data. In addition, the control unit 158 may output content such as quizzes, religious music, gymnastics, songs, and English listening, depending on the mood of the user.

In addition, the control unit 158 may perform real-time interactive communication with the user by analyzing the voice data of the user received via the microphone and outputting an appropriate response as voice data. At this time, the control unit 158 may recognize that an abnormality has occurred to the user if sensing data has not been collected within a threshold time based on a defined time, and send a message notifying that there is an emergency situation to the monitoring server 200 and the terminal device 300. Further, if the control unit 158 recognizes that an abnormality has occurred in the heart rate of the user, it may send a message notifying that there is an emergency situation to the monitoring server 200 and the terminal device 300.

The control unit 158 may transmit the sensing data and the like obtained by the second device 150 during the interactive communication with the user conducted when the user was taking in food such as a meal, snack, or the like, to the monitoring device 200 so that the monitoring device 200 can diagnose diseases, such as depression and dementia, in the user at an early stage.

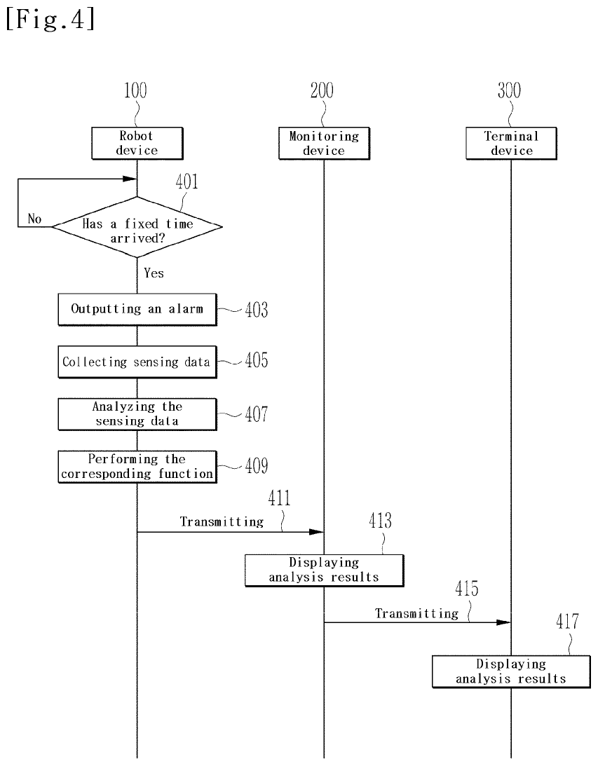

FIG. 4 is a flow diagram for describing a method of providing a food intake support service in accordance with an embodiment of the present disclosure.

Referring to FIG. 4, if the arrival of a preset fixed time is confirmed in step 401, the robot device 100 performs step 403, or continuously determines whether the preset fixed time has arrived if the arrival of the preset fixed time is not confirmed. In step 403, the robot device 100 outputs an alarm notifying that the fixed time has arrived. At this time, the preset fixed time may include a mealtime, a touch time at which the robot device 100 should be touched, a time at which the robot device 100 should sense the movement of the user, and the like.

In addition, the robot device 100 may output an alarm as voice data. For example, if the user is a male, the robot device 100 may output an alarm so that the user can take in food, such as “Grandpa, let's have breakfast together” and the like. At this time, the robot device 100 may output voice data, such as “Grandpa, the healthy meal we are having for lunch today includes barley rice, potato soup, diced radish kimchi, seasoned aster scaber, and bulgogi grilled with reduced salt. Having less rice and plenty of vegetables and fruits is beneficial for your health.”, “Grandpa, I am happiest when I eat with you. Please eat while talking with me.”, and the like.

In addition, the robot device 100 may output voice data that can lead the user to touch the robot device 100 or to move to the vicinity of the robot device 100, such as “Grandpa, please pet me,” “Grandpa, please play with me,” “Grandpa, I'm bored. Have a chat with me.”, and the like.

In step 405, the robot device 100 may collect sensing data. At this time, if the mealtime has arrived, the second device 150 included in the robot device 100 may obtain sensing data on the salinity, temperature, whether the food is spoiled, food intake speed, etc., of the food sensed by the first device 110 by using the plurality of sensors 111 included in the first device 110, and the second device 150 may obtain the sensing data.

Further, if the touch time or the time at which the movement should be sensed has arrived, the second device 150 may obtain at least one of biosignal data of the user, movement data of the user, image data including the face of the user, and voice data of the user as sensing data by using at least one of the sensor unit 153, the camera 154, and the microphone included in the second device 150.

In step 407, the robot device 100 analyzes the plurality of sensing data obtained by the first device 110 and the sensing data obtained by the second device 150 and performs step 409. In step 409, the robot device 100 performs the corresponding function. More specifically, the robot device 100 checks the mood of the user based on the sensing data obtained by the second device 150 when the mealtime has arrived, and checks the salinity, temperature, whether the food is spoiled, etc., of the food to be taken in by the user based on the plurality of sensing data obtained by the first device 110. Then, the robot device 100 may output voice data, such as “Grandpa, please chew all the side dishes thoroughly, just like I do. Enjoy your meal.”, “Grandpa, today's potato soup is not too salty and the seasoning is just right.”, “Grandpa, you had your meal slowly and chewed thoroughly for 30 minutes today. Please wash the spoon thoroughly and put it back in the storage case. Then it will be sterilized and dried cleanly. See you later.”, “Grandpa, it seems like you are eating too hastily today. Please take your time.” etc. In addition, the robot device 100 may perform real-time interactive communication between the user who is taking in food and the robot device 100 by analyzing the mood of the user checked based on the sensing data obtained by the second device 150 and the voice data of the user received via the microphone included in the second device 150 and outputting an appropriate response as voice data.

In addition, the robot device 100 checks at least one of the body temperature, presence or absence of movement, and mood of the user based on the sensing data obtained by the second device 150 when the touch time or the time at which the movement should be sensed has arrived. Then, the robot device 100 may output voice data, such as “Grandpa, your hands are too cold. Please put a hot pack on your body or drink a cup of warm tea.”, “Grandpa, why are you in a bad mood today? Then I feel bad too. Shall we listen to some cheerful songs?” etc. Next, the robot device 100 may output content such as quizzes, religious music, gymnastics, songs, and English listening according to a request by the user or conditions preset by the user. In addition, the robot device 100 may perform real-time interactive communication between the user and the robot device 100 by analyzing the mood of the user checked based on the sensing data obtained by the second device 150 and the voice data of the user received via the microphone and outputting an appropriate response as voice data.

In step 411, the robot device 100 transmits the analysis results of the plurality of sensing data obtained by the first device 110 that were analyzed in step 407 to the monitoring device 200. Further, in step 411, the robot device 100 may transmit the sensing data and the like obtained by the second device 150 during the interactive communication with the user conducted when the user was taking in food such as a meal, snack, or the like, to the monitoring device 200.

In step 413, the monitoring device 200 may display the average salinity value and average temperature value of the food, the eating time and the number of meals for the user, an AI statistical analysis report, and the like, as visual data based on the analysis results. Further, in step 413, the monitoring device 200 can diagnose diseases, such as depression and dementia, in the user at an early stage based on the sensing data obtained by the second device 150 during the interactive communication.

In step 415, the monitoring device 200 may transmit the visual data and the early diagnosis results to the terminal device 300, and in step 417, the terminal device 300 may display the visual data and the early diagnosis results received from the monitoring device 200.

In this way, even users who are not accustomed to using electronic devices such as smartphones or the like can seamlessly check their eating patterns when taking in food, etc., by using only the robot device 100. In addition, as the caregiver or welfare worker for the user can check the eating pattern or health condition of the user in real-time or periodically by using the terminal device 300, there is the effect of being able to check if an abnormality has occurred to the user without having to visit in person.

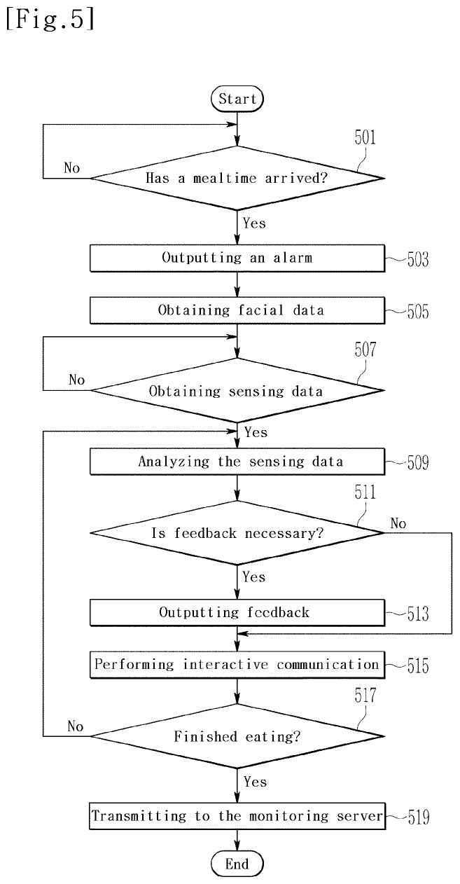

FIG. 5 is a flowchart for describing a method of providing a food intake support service by a second device included in a robot device in accordance with an embodiment of the present disclosure.

Referring to FIG. 5, in step 501, the control unit 158 checks whether a preset mealtime has arrived. As a result of checking in step 501, the control unit 158 performs step 503 if the preset mealtime has arrived, or the control unit 158 continuously checks whether the mealtime has arrived if the preset mealtime has not arrived.

In step 503, the control unit 158 outputs an alarm notifying that the mealtime has arrived as voice data. For example, if the user is a male, the robot device 100 may output an alarm so that the user can eat a meal, such as “Grandpa, let's have breakfast together” and the like. At this time, the robot device 100 may output voice data, such as “Grandpa, the healthy meal we are having for lunch today includes barley rice, potato soup, diced radish kimchi, seasoned aster scaber, and bulgogi grilled with reduced salt. Having less rice and plenty of vegetables and fruits is beneficial for your health.”, “Grandpa, I am happiest when I eat with you. Please eat while talking with me.”, and the like.

In step 505, the control unit 158 may obtain sensing data from at least one of the sensor unit 153, the camera 154, and the microphone. To this end, the control unit 158 may lead the user to the vicinity of the second device 150 so that the second device 150 can obtain sensing data on the user by outputting voice data, such as “Grandpa, I want to see your face. Please make eye contact with me.”, “Grandpa, I want to hear your voice.”, and the like.

In step 507, the control unit 158 checks whether a plurality of sensing data has been received from the first device 110. As a result of checking in step 507, the control unit 158 performs step 513 if the plurality of sensing data has been received from the first device 110, or waits to receive the plurality of sensing data if the plurality of sensing data has not been received. At this time, the plurality of sensing data may be obtained from the moment the user takes the first device 110 out of the storage case 110b, which can be checked with motion sensing data obtained from a motion sensor included in the first device 110. In this way, when it is confirmed from the motion sensor that the user has taken the first device 110 out of the storage case 110b, the control unit 158 may output a voice message, such as “Grandpa, please chew slowly and thoroughly and enjoy your meal.”, and the like.

In addition, the plurality of sensing data may include sensing data obtained from at least one of the motion sensor, the salinity sensor, the temperature sensor, or the gas sensor included in the first device 110. The plurality of sensing data may refer to sensing data that allows for checking the moving speed of the first device 110, the salinity, temperature, whether the food is spoiled, etc., of the food placed on the first device 110 when the user eats food using the first device 110.

In step 509, the control unit 158 performs an analysis of the plurality of sensing data received from the first device 110. More specifically, the control unit 158 may analyze the plurality of sensing data and check the eating time of the user based on the salinity and temperature of the food, whether the food is spoiled, and the moving speed of the first device 110. Further, the control unit 158 may check the mood of the user based on the sensing data obtained by the second device 150. In step 511, as a result of analyzing the plurality of sensing data received from the first device 110, the control unit 158 performs step 513 if it is determined that the user needs feedback regarding food intake, or performs step 515 if it is determined that feedback is not needed.

In step 515, the control unit 158 performs real-time interactive communication with the user who is taking in food by taking into account the mood of the user checked in step 509 and the voice data of the user received via the microphone, and then performs step 517. This may utilize functions such as AI (artificial intelligence) chatbots or the like.

Conversely, if it is determined that the user needs feedback while eating, the control unit 158 outputs feedback related to the conditions of the food being taken in and the eating speed as voice data in step 513, such as “Grandpa, please chew all the side dishes thoroughly, just like I do.”, “Grandpa, the potato soup is a bit salty today. Please add some warm water to the soup and take it.”, and the like. Next, in step 515, the control unit 158 may perform real-time interactive communication with the user who is taking in food by taking into account the mood of the user checked in real-time while the user is eating and the voice data of the user received via the microphone.

Next, in step 517, the control unit 158 performs step 519 if it is confirmed that the user has finished eating, or returns to step 509 and performs steps 509 to 515 again if it is confirmed that the user has not finished eating. In addition, the control unit 158 may confirm that the user has finished eating when no further movement is sensed by the first device 110 or when it is confirmed that the first device 110 is stored in the storage case 110b.

In this way, when it is confirmed that the user has finished eating, the second device 150 may output voice messages as feedback, such as “Grandpa, you ate too hastily today. Next time, have your meal slowly and chew thoroughly for 30 minutes. Please wash the spoon thoroughly and put it back in the storage case. Then it will be sterilized and dried cleanly. See you later.”, and the like.

In step 519, the control unit 158 may transmit the analysis results of the plurality of sensing data obtained by the first device 110 to the monitoring device 200 until the meal is finished. Thereby, the manager of the monitoring device 200 can check the average salinity value and average temperature value of the food taken in by the user, and can check and display the eating time and the number of meals for the user, an AI statistical analysis report, and the like, as visual data. Furthermore, in step 519, the control unit 158 may transmit the sensing data and the like obtained by the second device 150 during the interactive communication with the user to the monitoring device 200. Thereby, the manager of the monitoring device 200 can diagnose diseases, such as depression and dementia, in the user at an early stage.

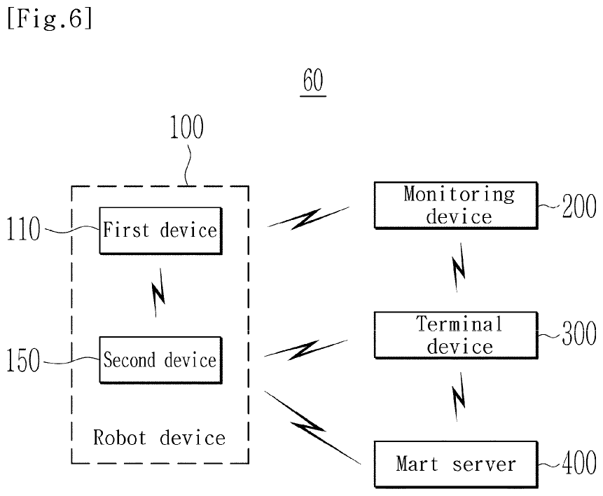

FIG. 6 is a diagram showing a system that provides a food intake support service in accordance with another embodiment of the present disclosure.

Referring to FIGS. 1 to 6, a system 60 in accordance with another embodiment of the present disclosure may include a robot device 100, a monitoring device 200, a terminal device 300, and a mart server 400.

Since the robot device 100, the monitoring device 200, and the terminal device 300 perform the same functions as the robot device 100, the monitoring device 200, and the terminal device 300 described in FIGS. 1 to 3, detailed descriptions thereof will be omitted.

However, in addition to FIGS. 1 to 3, the robot device 100 may transmit a purchase request signal for food ingredients to the mart server 400 via communication with the mart server 400. To this end, the robot device 100 may perform wireless communication with the mart server 400, such as 5G (5th generation communication), LTE (long term evolution), LTE-A (long term evolution-advanced), or Wi-Fi (wireless fidelity).

More specifically, when a meal plan guidance request signal is received, the robot device 100 presents a meal plan based on foods containing nutrients required by the user on the basis of the health condition of the user stored in the robot device 100. At this time, the meal plan may be a meal plan for a fixed period of time, for example, 3 days. In addition, the meal plan guidance request signal may be generated from input received from the user via the input unit 152 or the microphone, and may be based on confirming that the user has finished eating, as shown in FIG. 5. Further, the meal plan guidance request signal may be based on the arrival of a time preset by the user.

When a purchase request signal for ingredients to be purchased out of the ingredients for preparing for the meal plan is received, the robot device 100 transmits a purchase signal for purchasing the ingredients corresponding to the purchase request signal to the mart server 400. The robot device 100 automatically orders the ingredients corresponding to the purchase request signal via the mart server 400. At this time, the order quantity of the ingredients may be a minimum order quantity, and the order may be placed based on an order quantity preset by the user.

The robot device 100 may make a payment using a payment method that the user has stored in advance in the mart server 400 for payment, or may also make a payment via the terminal device 300 used by the caregiver of the user that has been stored in the mart server 400 in advance.

The mart server 400 is a device managed by a mart set in advance by the user who uses the robot device 100, the caregiver of the user, or the like, and may be an electronic device or a server such as a computer. The mart server 400 receives the purchase request signal for the food ingredients from the robot device 100. To this end, the mart server 400 may perform wireless communication with the robot device 100, such as 5G (5th generation communication), LTE (long term evolution), LTE-A (long term evolution-advanced), or Wi-Fi (wireless fidelity).

The mart server 400 performs delivery of the food ingredients to the address where the ingredients were ordered once payment for the order of the food ingredients corresponding to the purchase request signal requested from the robot device 100 is completed. At this time, the payment and the address may be inputted and preset by any of the robot device 100 and the terminal device 300.

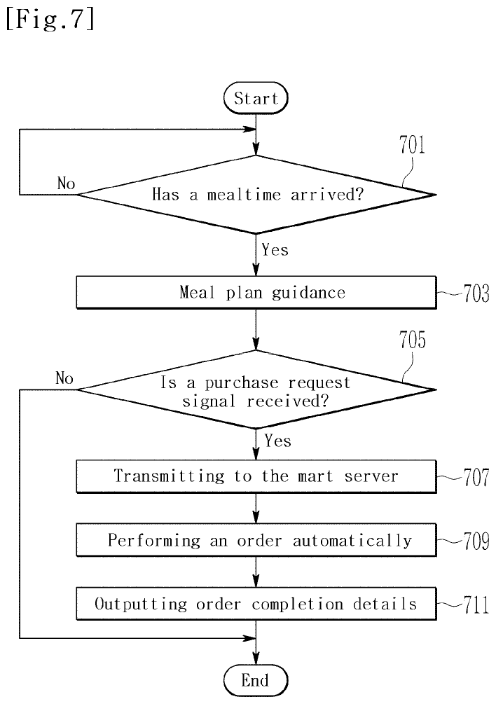

FIG. 7 is a flowchart for describing a method of providing a food intake support service by a second device included in a robot device in accordance with another embodiment of the present disclosure.

Referring to FIG. 7, in step 701, the control unit 158 checks whether a meal plan guidance request signal has been received. As a result of checking in step 701, the control unit 158 performs step 703 if the meal plan guidance request signal has been received, or the control unit 158 waits to receive the meal plan guidance request signal if the meal plan guidance request signal has not been received. At this time, the meal plan guidance request signal may be generated from input received from the user via the input unit 152 or the microphone, and may be based on confirming that the user has finished eating, as shown in FIG. 5. Further, the meal plan guidance request signal may be based on the arrival of a time preset by the user.

In step 703, the control unit 158 presents a meal plan based on foods containing nutrients required by the user on the basis of the health condition of the user stored in the memory 157. At this time, the meal plan may be a meal plan for a fixed period of time, for example, 3 days.

In step 705, the control unit 158 performs step 707 if a purchase request signal for at least one of the ingredients necessary for preparing for the meal plan is received from the user via the input unit 152 or the microphone, or ends the corresponding process if the purchase request signal is not received. In step 707, the control unit 158 transmits a purchase signal for purchasing the ingredients corresponding to the purchase request signal to the mart server 400. At this time, the mart server 400 may be a server operated by a mart preset by the user.

In step 709, the control unit 158 automatically orders the ingredients corresponding to the purchase request signal at the mart server 400. At this time, the order quantity of the ingredients may be a minimum order quantity, and the order may be placed based on an order quantity preset by the user. Next, in step 711, the control unit 158 outputs a message notifying that the order has been completed. In this case, the user may store a means for payment in advance in the mart server 400, and payment may also be made via the terminal device 300 used by the caregiver of the user.

The devices and control thereof described above may be implemented in hardware components, software components, and/or combinations of hardware components and software components. For example, the devices and components described in the embodiments may be implemented using one or more general-purpose computers or special-purpose computers, such as processors, controllers, ALUs (arithmetic logic units), digital signal processors, microcomputers, FPGAs (field programmable gate arrays), PLUS (programmable logic units), microprocessors, or any other devices capable of executing and responding to instructions. A processing device may execute an operating system (OS) and one or more software applications running on the operating system. Further, the processing device may access, store, manipulate, process, and generate data in response to the execution of software. For case of understanding, although there are cases described where a single processing device is used, those having ordinary skill in the art will appreciate that the processing device may include a plurality of processing elements and/or a plurality of types of processing elements. For example, the processing device may include a plurality of processors or a single processor and one controller. In addition, other processing configurations, such as parallel processors, are also possible.

Software may include computer programs, code, instructions, or combinations of one or more of these, and may configure the processing device to operate as desired or may instruct the processing device independently or in combination. The software and/or data may be embodied permanently or temporarily in any type of machine, component, physical device, virtual device, and computer storage medium or device in order to be interpreted by the processing device or to provide instructions or data to the processing device. The software may be distributed over networked computer systems and thus stored or executed in a distributed manner. The software and data may be stored on one or more computer-readable recording media.

The methods in accordance with the embodiments may be implemented in the form of program instructions that can be executed via a variety of computer means and may be recorded on a computer-readable medium. The computer-readable medium may include program instructions, data files, data structures, and the like, alone or in combination. The program instructions to be recorded on the medium may be those specially designed and configured for the embodiments, or those known and available to a person of ordinary skill in the art of computer software. Examples of the computer-readable recording medium include magnetic media such as hard disks, floppy disks, and magnetic tapes, optical media such as CD-ROMs and DVDs, magneto-optical media such as floptical disks, and hardware devices specially configured to store and execute program instructions, such as ROM, RAM, flash memory, etc. Examples of the program instructions include not only machine language code, such as those created by a compiler, but also high-level language code that can be executed by a computer using an interpreter or the like. The hardware devices described above may be configured to operate as one or more software modules, and vice versa, to carry out the operations of the embodiments.

As described above, although the embodiments have been described by way of limited embodiments and drawings, those having ordinary skill in the art can make various modifications and variations from the above description. For example, appropriate results can be achieved even if the described techniques are performed in a different sequence from the described methods, and/or the components of the described systems, structures, devices, circuits, and the like are incorporated or combined in a form different from the described methods, or are replaced or substituted by other components or equivalents.

Therefore, other implementations, other embodiments, and equivalents of the claims also fall within the scope of the claims set forth below.

What is claimed:

1. A method of providing a food intake support service, comprising:
outputting, by a second device included in a robot device comprising the second device and a first device, an alarm when a preset food intake time arrives;
receiving, by the second device, a plurality of sensing data related to food intake from the first device;
analyzing, by the second device, the plurality of sensing data;
outputting, by the second device, feedback based on the plurality of sensing data; and
performing, by the second device, a real-time interactive communication based on inputted voice data.

2. The method of claim 1, further comprising, after the outputting of the alarm:
obtaining, by the second device, sensing data on a user of the robot device; and
checking, by the second device, a mood of the user based on the sensing data obtained by the second device.

3. The method of claim 2, wherein the real-time interactive communication is performed based on the mood of the user that has checked based on the plurality of sensing data obtained by the second device and the voice data.

4. The method of claim 1, wherein the plurality of sensing data is received from a plurality of sensors including at least one of a motion sensor, a salinity sensor, a gas sensor, or a temperature sensor that is provided in the first device that is a cutlery including a spoon.

5. The method of claim 4, wherein the feedback is output based on at least one of a mastication speed of a user of the robot device, a salinity level contained in a food, whether the food is spoiled, or a temperature of the food based on the plurality of sensing data.

6. The method of claim 1, further comprising:
checking, by the second device, whether the food intake has ended from the first device; and
transmitting, by the second device, an analysis result obtained by analyzing the plurality of sensing data to a monitoring device.

7. The method of claim 6, further comprising, after the checking of whether the food intake has ended,
creating and outputting, by the second device, a meal plan for a fixed period of time based on a health condition of a user of the robot device.

8. The method of claim 7, further comprising, after the creating and outputting of the meal plan:
receiving, by the second device, a purchase request signal for at least one of ingredients required for the meal plan from the user; and
ordering ingredients according to the purchase request signal to a preset mart server.

9. A device configured to provide a food intake support service, comprising:
a first device comprising a plurality of sensors and configured to obtain a plurality of sensing data related to food intake; and
a second device configured to output an alarm when a preset food intake time arrives, analyze the plurality of sensing data received from the first device and output feedback, and perform a real-time interactive communication based on voice data inputted from a user.

10. The device of claim 9, wherein the second device is configured to check a mood of the user based on sensing data on the user and performs the real-time interactive communication based on the mood of the user and the voice data.

11. The device of claim 9, wherein the first device is a cutlery comprising a spoon and is provided with the plurality of sensors comprising at least one of a motion sensor, a salinity sensor, a gas sensor, or a temperature sensor.

12. The device of claim 11, wherein the second device is configured to output the feedback based on at least one of a mastication speed of the user, a salinity level contained in a food, whether the food is spoiled, or a temperature of the food based on the plurality of sensing data.

13. The device of claim 9, wherein the second device is configured to transmit, to a monitoring device, an analysis result obtained by analyzing the plurality of sensing data upon checking from the first device whether the food intake has ended.

14. The device of claim 13, wherein the second device is configured to create and output a meal plan for a fixed period of time based on a health condition of the user.

15. The device of claim 14, wherein the second device is configured to order ingredients according to a purchase request signal to a preset mart server in accordance with the purchase request signal for at least one of ingredients required for the meal plan received from the user.
